(19) United States
(12) Patent Application Publication
(10) Pub. No.: US 2025/0000656 A1
(43) Pub. Date: Jan. 2, 2025

(54) BONE JOINT IMPLANTS

(71) Applicant: Loci Orthopaedics Limited, Galway (IE)

(72) Inventors: Gerry CLARKE, Galway (IE); Fiona MANGAN, Galway (IE); Brendan BOLAND, Galway (IE)

(73) Assignee: Loci Orthopaedics Limited, Galway (IE)

(21) Appl. No.: 18/688,242

(22) PCT Filed: Aug. 23, 2022
(86) PCT No.: PCT/EP2022/073522

§ 371 Date: Feb. 29, 2024

(30) Foreign Application Priority Data
Sep. 2, 2021 (EP) 21194627.2

(51) Int. Cl. A61F 2/30; A61F 2/42

(52) U.S. Cl. CPC A61F 2/30771; A61F 2/4241; A61F 2002/30004

(57) ABSTRACT

An intramedullary stem (1) is for a CMC joint implant. It has a distal bone-engagement end (10) and a proximal end (11) for engaging an articulation component such as a ball to complete a ball-and-socket coupling to a trapezium-contacting platform. The stem has an integral body (2) of material with an external surface for engaging a bone and with an aperture (3) leading to a socket (4) in the proximal end for engaging an articulation component ball or the like. The stem comprises a proximally extending projecting portion 5) which partly surrounds the socket. It is of PEEK material, which provides the desired strength and bone-contacting properties. The stem may have a coating over an elongate portion (203) of the surface extending distally and also in a ring (204) around the proximal end, to achieve optimum adhesion without excessive difficulty in extraction during revision surgery.

### INTRODUCTION

The present invention relates to bone joint implants.

Degenerative arthritis such as osteoarthritis is generally treated with the surgical implantation of orthopaedic implants that are designed to restore the natural function of the joint. Most of these implants are made of metallic and polymer components.

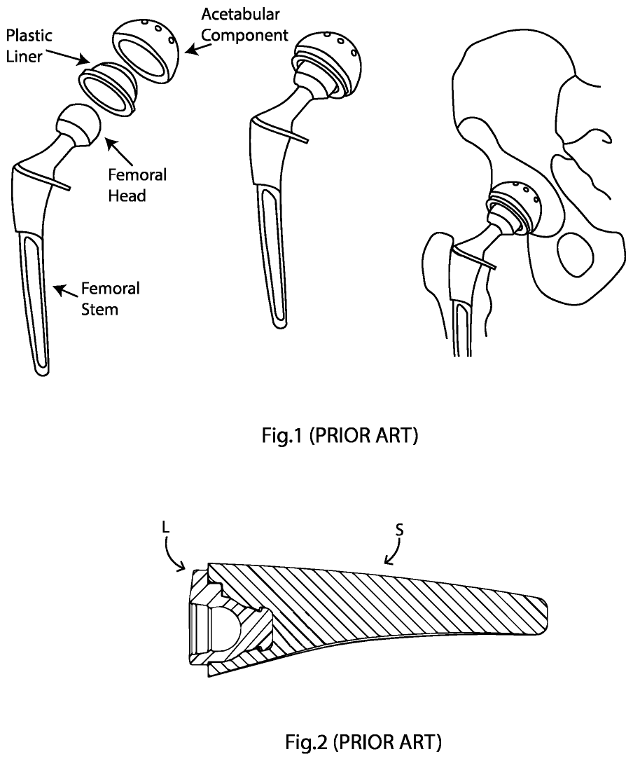

The metallic components provide rigidity for fixation into a bone such as the femur or metacarpal. In most cases, a polymer bearing surface is utilised to provide a smooth resilient bearing surface between two or more metallic components. An example is a hip joint implant consisting of a metal cup which is inserted into the pelvic acetabulum, a polymer liner with a socket, and a metallic head/stem which is seated in the femur, as illustrated in FIG. 1.

It is known that metal-on-metal joint implant components have had poor clinical outcomes due to the fact that metal-on-metal bearing surfaces can generate excessive metallic debris, which in turn causes a local and system inflammatory reaction. Therefore, the commonest material combination in orthopaedic implant design remains metal-polymer-metal.

The following documents describe implants in this field: WO2020/193078 (Loci), U.S. Pat. No. 3,760,427 (Schultz), US2009/216333 (Wolfe), GB1461154 (Miller), U.S. Pat. No. 3,909,853 (Lennox), EP1402854 (Depuy), US2013/338784) (Pallia), and FR3027213 (Groupe Lepine). In the example of WO2020/193078 shown in FIG. 2 an example illustration, of an implant with a metacarpal stem, having a main stem body S of metal and a liner insert L with a socket for an articulation component. This document also mentions that the features of the insert may be included in an integral manner.

The present invention is directed towards providing an implant of simpler construction.

### SUMMARY OF THE INVENTION

We describe a unitary intramedullary stem for a bone joint implant, the stem comprising an integral body with a distal bone-engagement end with a bone-contacting surface and a proximal end with an articulation feature configured for engaging an external articulation component to form an articulation coupling.

In one example, the proximal end comprises an aperture leading to the articulation feature. In one example, said articulation feature comprises a socket. In one example, the articulation feature comprises a ball for engaging an external articulation component socket or the like.

In one example, the stem is configured for engagement of the distal end in a metacarpal bone.

In one example, the stem comprises a proximally extending projecting portion which fully or partly surrounds the articulation component. In one example, said projecting portion forms an integral shoulder of the stem proximal end. In one example, thee stem comprises a plurality of articulation components in the proximal end.

In one example, the stem integral body comprises a polymer material. In one example, the material comprises polyether ether ketone, PEEK. In one example, the material comprises carbon fibre polyether ether ketone, CF-PEEK.

In one example, the stem comprises a coating on an external bone-contacting surface of said integral body. In one example, the external bone-contacting interface has surface roughness to promote an interference fit of the distal end. In one example, the bone-contacting surface comprises ridges, and/or pores, and/or fenestrations, and/or barbs, and/or interconnected lattices. In one example, the integral body is treated to have a surface roughness Ra greater than 6.0 μm before deposition of the coating.

In one example, the coating has a thickness of 254 μm+/−127 μm.

In one example, the coating comprises titanium plasma material, preferably CPTi. In one example, the coating is not applied over all of the bone-contacting surface of the integral body. Preferably, the coating is applied in an elongate region extending distally.

In one example, the coating includes a circumferential pattern extending around the integral body at or adjacent to the proximal end.

In one example, at least some of a bone-contacting surface of the stem has a surface roughness Ra in the range 20 μm to 40 μm.

We also describe an implant comprising an intramedullary stem of any example described herein and an external articulation component for engaging with the articulation component of the stem.

In one example, the stem comprises a ball socket in its proximal end and the articulation component comprises a ball on a neck, the ball being configured to fit in the socket.

We also describe a unitary intramedullary stem for a bone joint implant, the stem comprising an integral body with a distal bone-engagement end with a bone-contacting surface and a proximal end with an articulation feature configured for engaging an external articulation component to form an articulation coupling.

Preferably, the proximal end comprises an aperture leading to the articulation feature. Preferably, said articulation feature comprises a socket. Preferably, the proximal end articulation feature comprises a ball for engaging an external articulation component socket or the like.

Preferably, the stem is configured for engagement of the distal end in a metacarpal bone. Preferably, the stem comprises a proximally extending projecting portion which fully or partly surrounds the articulation component.

Preferably, said projecting portion forms an integral shoulder of the stem proximal end.

In some examples, the stem comprises a plurality of articulation components in the proximal end.

Preferably, the stem comprises a polymer material, and said the material may comprise PEEK.

In some examples, the stem comprises a coating on an external bone-contacting surface of said integral body.

Preferably, the external bone-contacting interface has surface roughness to promote an interference fit of the distal end and to promote osteointegration, and said surface may comprise ridges, and/or pores, and/or fenestrations, and/or barbs, and/or interconnected lattices.

We also describe an implant comprising an intramedullary stem of any example and an external articulation component for engaging with the articulation component of the stem.

### DETAILED DESCRIPTION OF THE INVENTION

The invention will be more clearly understood from the following description of some embodiments thereof, given by way of example only with reference to the accompanying drawings in which:

FIG. 1 is a set of views representative of a femoral implant of the prior art, and FIG. 2 is a sectional side view of components of a CMC implant of the prior art, both as referenced in the Introduction above;

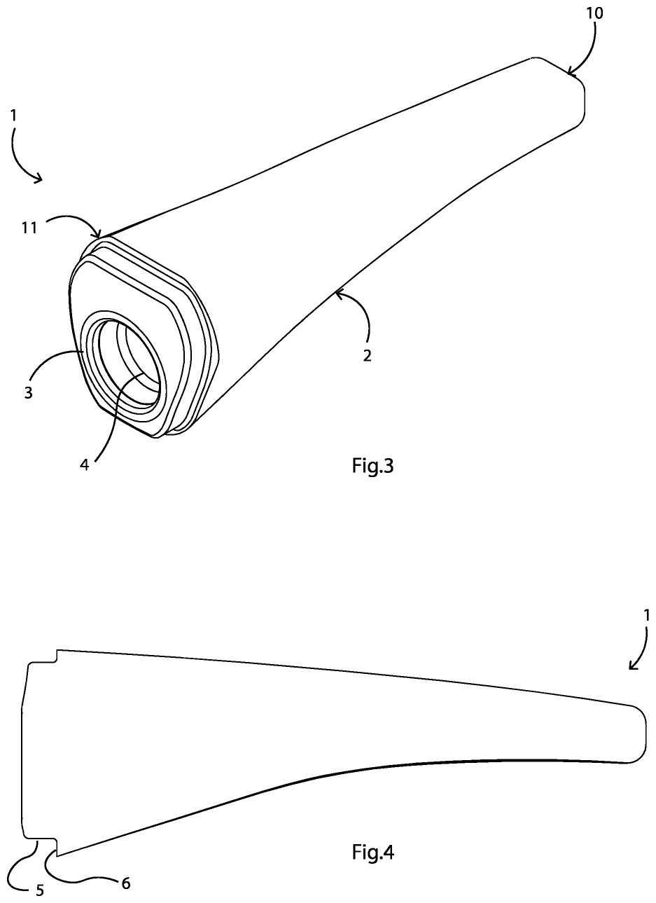

FIG. 3 is a perspective view of an intramedullary stem of the invention for a bone joint implant;

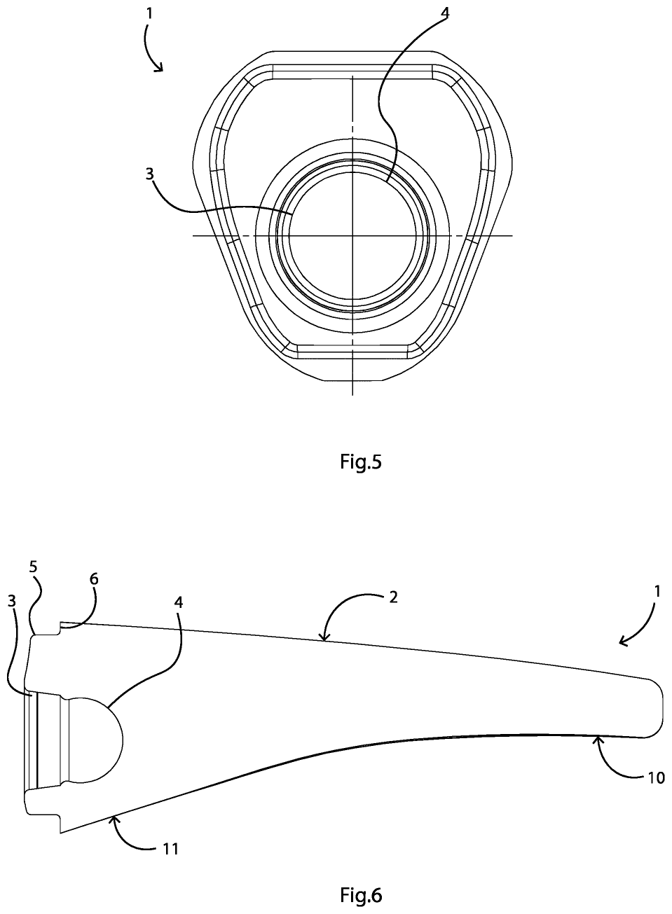

FIG. 4 is a side view, and FIG. 5 is and end view from the proximal end;

FIG. 6 is a diagrammatic side sectional view of the stem;

FIGS. 7 to 9 are a series of sketches showing an alternative implant used with replacement of the trapezium;

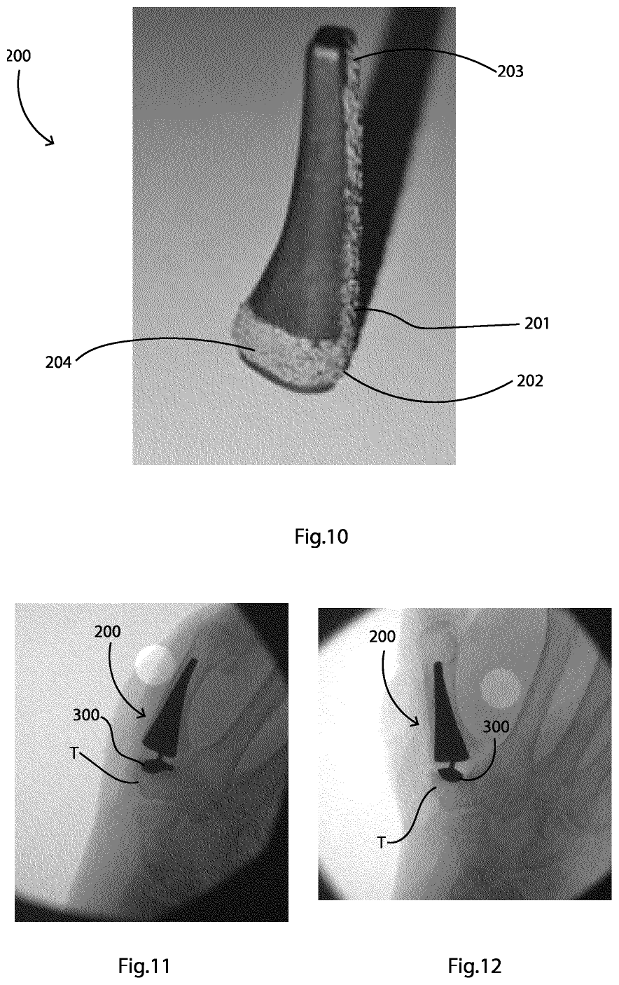

FIG. 10 is an image of another stem of the invention; and

FIGS. 11 and 12 are X-ray images showing a CMC joint implant in place in a patient, the implant having a stem and an integral saddle for sliding on the trapezium and a ball engaged in the socket of the stem.

Referring to FIGS. 3 to 6 an intramedullary stem 1 of the invention is in this case for a thumb carpometacarpal (CMC) joint. However, in other examples the invention provides a stem for another type of implant such as a hip implant of the type illustrated in FIG. 1. The stem 1 is for a CMC joint implant. It has a distal metacarpal bone-engagement end 10 and a proximal end 11 for engaging an articulation component such as a ball to complete a ball-and-socket coupling to a trapezium-contacting platform.

The stem 1 is a unitary component with an integral body 2 having a narrow distal end of the known configuration for a metacarpal stem, and a wider proximal end. The general shape is the known shape for a metacarpal stem, and it may vary according to the desired end use age group.

The stem body 2 has a narrow distal end 10 and a wider proximal end 11 which has an integral articulation feature, in this case an aperture 3 leading to a spherical socket 4. The proximal end of the body 2 comprises a proximally extending projection 5 which forms a shoulder in relation to the wider portion 6 of the body 2 immediately distal of the portion 5. The projecting portion 5 surrounds the aperture 3.

In one preferred example, the stem 1 comprises a PEEK polymeric material which provides a rigidity similar to that of the bone in which it is being implanted. In other examples, the stem may be metallic, which provides an increased rigidity. In each case, the head, which engages the stem to complete the implant, may be fabricated from an alternative material, for example a PyC (Pyrolytic Carbon) material which may have a similar Youngs Modulus to bone for use in a hemiarthroplasty where the head articulates directly on bone, used in combination with a PEEK stem. Alternatively, there may be a PEEK head in combination with a metallic stem.

The material of the stem 1 is preferably a polymer, and polyether ether ketone (PEEK) is particularly preferred. LSG (Life Science Grade) PEEK has the following properties in a preferred example:

| | |
|---|---|
| Rockwell Hardness | 105. |
| Tensile Strength | 115 MPa. |
| Young's Modulus | 4300 MPa. |

PEEK provides sufficient stem rigidity while simultaneously providing a good smooth bearing surface. Benefits of PEEK include that it may be Gamma sterilized and that it can be surface roughened. There may be a TPS (Titanium Plasma Spray) coating or a HA (Hydroxyapatite) coating, each of which is particularly advantageous. The latter fortifies the bone-implant interface and provides a porous surface to promote osteointegration.

Surface roughening provides natural contours on the surface into which bone can grow, further integrating the implant into the position over the geometrical positioning of the implant. A TPS coating provides a roughened surfaces that aids in the interference fit of the implant into bone, and to a lesser extent, osteointegration. Hydroxyapatite coating also improves the interference fit of an implant stem into the bone as well as allowing for some osteointegration.

In other examples the stem body material may include Carbon fibre reinforced PEEK (CF-PEEK), giving higher strength mechanical properties where it is required.

| | |
|---|---|
| Rockwell Hardness | 102 |
| Tensile Strength | 144 MPa |
| Young's Modulus | 9200 MPa |

Other suitable materials are for example pyrocarbon and graphine.

Functional gradient materials may be employed, wherein properties of the implant may be varied at different locations on or within the implant in order to optimise the functionality of the implant at each of those locations. Processes which may be employed to achieve this include additive manufacturing (AM), chemical vapour deposition (CVD), laser metal deposition (LMD), laser engineered net shaping (LENS), direct metal deposition (DMD), electron beam direct manufacturing (EBDM) and sintering.

Materials with a stiffness in excess of bone can cause remodeling of bone through Wolff forces, but the use of a material which has a rigidity closer to bone can avoid this.

The external bone-contacting interface preferably, in various examples, has surface roughness to promote an interference fit of the distal end. It may for example include any or all of ridges, and/or pores, and/or fenestrations, and/or barbs, and/or interconnected lattices.

The stem external bone-contacting surface may be ridged for desired strength properties.

Enhanced performance for a polymeric integral stem can be gained by using “pitch” type PEEK blended with short carbon fibers, such as Invibio's OPTIMA™ carbon fiber-PEEK (CF-PEEK) material.

In one preferred example, CF-PEEK material is spray coated with a titanium plasma coating, for example with commercially pure titanium (CPTi) to provide an interference fit and promote osseointegration. For this coating, the CF-PEEK substrate is preferably grit-blasted with #36 size grit to yield a surface roughness greater Ra greater than 6.0 μm before application of the coating.

The coating mesh powder is preferably −200 to +325 (CPTi Sponge) and the coating thickness is preferably 254 μm+/−127 μm.

The surface of the coating, which engages the bone has a surface roughness of 30 μm, and more generally it is preferred that it be in the range of 20 μm to 40 μm. This is particularly effective for osteointegration.

The coating is preferably not applied over all of the bone-contacting surface of the integral body. A complete surface coating would make the implant difficult to extract, and only partial distribution of the coating provides the desired adherence benefits from the coating without making the stem too difficult to extract. The preferred coating distribution is along a line extending distally, such as along a dorsal segment of the stem as shown in FIG. 10. In this example an integral stem 200 with a CF-PEED body has a CPTi coating as described above extending distally in an elongate pattern 201 from a proximal end 202 to a distal end 203, although in other examples this elongate pattern does not extend the full length of the stem. It is also preferable that there be a continuous band around the proximal base area to combat hoop stress, and this is sown as a ring 204 in FIG. 10.

### Advantages

Utilizing a polymeric integral stem provides a mechanism to locate a bearing surface socket closer to an edge of a stem than would be the case with a separate liner. Similarly, a larger socket may be provided than would be possible in the case of a separate liner. To illustrate these points reference is again made to FIG. 6. The socket 4 of this drawing may alternatively be located at a position much closer to the top shoulder 6. This would not be possible because of the minimum wall thickness required for a liner insert.

Because there is only one component, manufacturing errors are less likely, as are risks of infections. There is no liner-stem body interface to provide risk of backside wear. There is no risk of disassembly or levering of a liner from a stem main body, no galvanic corrosion, no risk of metal-on-metal contact with resultant debris generation. Also, there is more flexibility in manufacturing.

Another advantage is that in the case of a revision surgery, the stem may be removed in one piece, rather than separate tasks of removal of different parts with different tools, such as a polymeric component and a metallic component. A one-piece stem such as this can be removed using one set of tools and in one surgical action, minimizing the risk of complications, and the overall surgical complexity or a revision procedure.

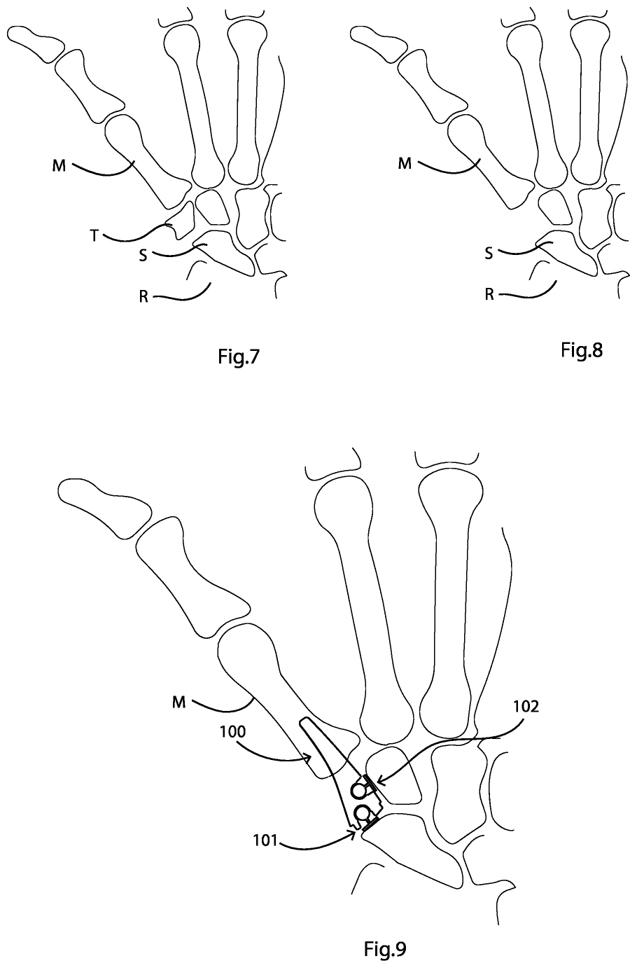

Another advantage is that there may be two or more articulating surfaces such as sockets or male components. For example, where multiple bones are articulating with an implant concurrently, only one stem may be necessary to provide the anchoring structure, without the need for individual polymeric bearing surfaces. As an example, FIG. 7 shows bones of the hand, metacarpal M, Trapezium T, Scaphoid S, and Radius R. FIG. 8 shows the situation after removal of the trapezium, and FIG. 9 shows diagrammatically the situation after insertion of a unitary stem 100 into the metacarpal and replacing the trapezium, with two articulating couplings 101 and 102, the coupling 101 being with the Scaphoid and the coupling 102 being with the adjoining Trapezium.

Another advantage is that there is no assembly of the stem necessary before or during surgery, avoiding risk of human error with the wrong, or mis-sized components being inadvertently assembled.

Because the socket is integrated within the stem there is excellent flexibility in its location. The socket size is only limited by external dimensions of the stem. The central axis of the socket may be placed in any location that optimizes the biomechanics of the implant construct without the limitation of a distinct polymeric Liner structure.

Other advantages are simple sterilization, shortened manufacturing time, suitability for 3D printing methods, Also, the stem can be 3D printed and the socket machined at a later date.

Components of embodiments can be employed in other embodiments in a manner as would be understood by a person of ordinary skill in the art. The invention is not limited to the embodiments described but may be varied in construction and detail.

1. A unitary intramedullary stem for a bone joint implant, the stem comprising an integral body with a distal bone-engagement end with a bone-contacting surface and a proximal end with an articulation feature configured for engaging an external articulation component to form an articulation coupling.

2. The stem of claim 1, wherein the proximal end comprises an aperture leading to the articulation feature.

3. The stem of claim 1, wherein the articulation feature comprises a socket.

4. The stem of claim 1, wherein the proximal end articulation feature comprises a ball for engaging an external articulation component socket or the like.

5. The stem of claim 1, wherein the stem is configured for engagement of the distal end in a metacarpal bone.

6. The stem of claim 1, wherein the stem comprises a proximally extending projecting portion which fully or partly surrounds the articulation component, and the projecting portion forms an integral shoulder of the stem proximal end.

7. (canceled)

8. The stem of claim 1, further comprising a plurality of articulation components in the proximal end.

9. The stem of claim 1, wherein the stem integral body comprises a polymer material.

10. The stem of claim 9, wherein the material comprises polyether ether ketone (PEEK).

11. The stem of claim 10, wherein the material comprises carbon fibre polyether ether ketone (CF-PEEK).

12. The stem of claim 1, wherein the stem comprises a coating on an external bone-contacting surface of the integral body.

13. The stem of claim 1, wherein the external bone-contacting surface has surface roughness to promote an interference fit of the distal end.

14. The stem of claim 12, wherein the bone-contacting surface comprises ridges, pores, fenestrations, barbs, interconnected lattices, or combinations thereof.

15. The stem of claim 12, wherein the integral body is treated to have a surface roughness Ra greater than 6.0 μm before deposition of the coating.

16. The stem of claim 12, wherein the coating has a thickness of 254 μm+/−127 μm.

17. The stem of claim 12, wherein the coating comprises titanium plasma material.

18. The stem of claim 12, wherein the coating is not applied over all of the bone-contacting surface of the integral body, and the coating is applied in an elongate region extending distally.

19. (canceled)

20. The stem of claim 18, wherein the coating includes a circumferential pattern extending around the integral body at or adjacent to the proximal end.

21. (canceled)

22. An implant comprising:
an intramedullary stem, the intramedullary stem comprising an integral body with a distal bone-engagement end with a bone-contacting surface and a proximal end with an articulation feature configured for engaging an external articulation component to form an articulation coupling, and
an external articulation component for engaging with the articulation component of the stem.

23. The implant of claim 22, wherein the proximal end of the stem comprises a ball socket and the articulation component comprises a ball on a neck, the ball being configured to fit in the socket.
